(19) United States
(12) Patent Application Publication
(10) Pub. No.: US 2026/0325431 A1
(43) Pub. Date: Oct. 22, 2026

(54) COMPOSITION COMPRISING HYDROGEL

(71) Applicant: Amorepacific Corporation, Seoul (KR)

(72) Inventors: Youngsuk CHO, Yongin-si (KR); Myeongjin GOH, Yongin-si (KR); Hyungmin KIM, Yongin-si (KR); Dajeong LEE, Yongin-si (KR); Young Gyu KANG, Yongin-si (KR); Sungyeon CHO, Yongin-si (KR); Jeongah HWANG, Yongin-si (KR)

(73) Assignee: Amorepacific Corporation, Seoul (KR)

(21) Appl. No.: 19/632,676

(22) Filed: Mar. 30, 2026

(30) Foreign Application Priority Data
Apr. 16, 2025 (KR) 10-2025-0049703

(51) Int. Cl. A61K 8/90; A61K 8/02; A61Q 19/08

(52) U.S. Cl. CPC A61K 8/90; A61K 8/0241; A61Q 19/08; A61K 2800/805; A61K 2800/81; A61K 2800/95

(57) ABSTRACT

The present specification discloses a composition comprising a cross-linked gel in which gamma-polyglutamic acid or a salt thereof, and hyaluronic acid or a salt thereof are cross-linked in a weight ratio of more than 0.65 and less than 2:1. The present disclosure may provide useful efficacy to skin while maintaining excellent biocompatibility of the two components by mixing gamma-polyglutamic acid or a salt thereof with hyaluronic acid or a salt thereof, and providing the components in the form of a cross-linked gel photo-cross-linked without a cross-linking agent after manufacturing the components into each gel form.

### CROSS REFERENCE TO RELATED APPLICATION

The present application claims priority to Korean Patent Application No. 10-2025-0049703, filed Apr. 16, 2025, the entire contents of which are hereby incorporated by this reference.

### BACKGROUND OF THE INVENTION

### Field of the Invention

A composition comprising a hydrogel is disclosed in the present specification.

### Description of the Related Art

Gamma-polyglutamic acid is a gamma-polypeptide connected by an amide bond between alpha-amino and gamma-carboxylic acid groups, and is mostly biosynthesized by Bacillus species microorganisms. For gamma-polyglutamic acid, since it is excellent in biocompatibility and has an excellent moisture retention capacity, attempts are being made to use such in various industrial fields such as food, medicine, and cosmetics by manufacturing such into a cross-linked gel. However, despite the excellent biocompatibility of gamma-polyglutamic acid itself, due to a cross-linking agent such as polyethylene glycol used to provide this in a hydrogel form by mixing this with other components, there is a possibility of inducing skin irritation and allergic reactions, and thus there is a problem that consumer reluctance is high. Accordingly, development for a new type of cross-linked gel capable of simultaneously providing efficacy and stability of gamma-polyglutamic acid is required.

### SUMMARY OF THE INVENTION

A problem to be solved by the present disclosure is to provide a composition of a hydrogel formulation capable of simultaneously providing efficacy and stability of gamma-polyglutamic acid and a manufacturing method thereof.

To solve the above problem, one embodiment of the present disclosure provides a composition comprising, as an active ingredient, a cross-linked gel in which gamma-polyglutamic acid or a salt thereof, and hyaluronic acid or a salt thereof are cross-linked in a weight ratio of more than 0.65 and less than 2:1.

In addition, one embodiment of the present disclosure provides, as a manufacturing method of the composition, a manufacturing method comprising a cross-linked gel manufacturing step comprising photo-cross-linking a gel of gamma-polyglutamic acid or a salt thereof, and a gel of hyaluronic acid or a salt thereof at a weight ratio of gamma-polyglutamic acid or a salt thereof: hyaluronic acid or a salt thereof of more than 0.65 and less than 2:1.

The present disclosure is related to a composition comprising a cross-linked gel in which gamma-polyglutamic acid or a salt thereof, and hyaluronic acid or a salt thereof are cross-linked in a weight ratio of more than 0.65 and less than 2:1. The present disclosure may provide useful efficacy to skin while maintaining excellent biocompatibility of the two components by mixing gamma-polyglutamic acid or a salt thereof with hyaluronic acid or a salt thereof, and providing the components in the form of a cross-linked gel photo-cross-linked without a cross-linking agent after manufacturing the components into each gel form.

### BRIEF DESCRIPTION OF THE DRAWINGS

FIG. 1 is a view showing a result of analyzing a particle size distribution of a cross-linked gel used in a composition according to one embodiment of the present disclosure.

FIG. 2 is a view showing a result of analyzing a relative amount of CASP14 gene expression according to a weight ratio of gamma-polyglutamic acid (PGA) and hyaluronic acid (HA) as one embodiment of the present disclosure, in which CTL means an untreated group, EGCG means a control group, PGA:HA 1:1.5 means Comparative Example 1, 1:1 means Example 1, 2:1 means Comparative Example 2, 4:1 means Comparative Example 3, and 6:1 means Comparative Example 4.

FIG. 3 is a view showing a comparative analysis of a viscosity coefficient G″ of Example 1 (HA:PGA=1:1 mixture) and Comparative Example 5 (HA cross-linked gel alone) according to one embodiment of the present disclosure.

FIG. 4 is a view showing a comparative analysis of a feeling of use of Example 1 (HA:PGA=1:1 mixture) and Comparative Example 5 (HA cross-linked gel alone) according to one embodiment of the present disclosure.

FIG. 5 is a view showing a comparative analysis of a skin moisturizing efficacy of Example 2 (HA:PGA=1:1 mixture), Comparative Example 5 (HA cross-linked gel alone), and Comparative Example 6 (PGA cross-linked gel alone) according to one embodiment of the present disclosure.

### DETAILED DESCRIPTION OF THE INVENTION

Hereinafter, exemplary embodiments of the present disclosure will be described in detail with reference to the accompanying drawings.

The embodiments of the present disclosure disclosed herein are illustrated for purposes of description only, and the embodiments of the present disclosure may be practiced in various forms and should not be interpreted as limiting to the embodiments described herein. The present disclosure is subject to various modifications and may have various forms, and the embodiments are not intended to limit the present disclosure to any particular disclosure form, but are to be understood to include all modifications, equivalents, or substitutions that fall within the scope of the spirit and art of the present disclosure. Singular expressions include plural expressions unless clearly described as different meanings in the context. In the present application, the terms “comprises,” “comprising,” “includes,” “including,” “containing,” “has,” “having” or other variations thereof are inclusive and therefore specify the presence of stated features, integers, steps, operations, constituent elements, components (or parts), and/or combinations thereof, but do not exclude the presence or addition of one or more other features, integers, steps, operations, constituent elements, components (or parts), and/or combinations thereof.

One embodiment of the present disclosure may provide a composition comprising, as an active ingredient, a cross-linked gel in which gamma-polyglutamic acid or a salt thereof, and hyaluronic acid or a salt thereof are cross-linked in a weight ratio of more than 0.65 and less than 2:1.

In the present disclosure, “gamma-polyglutamic acid (γ-polyglutamic acid, γ-PGA)” is a homopolymer connected by an amide linkage between alpha-amino and gamma-carboxylic acid groups. Gamma-polyglutamic acid may be mainly biosynthesized by various types of microorganisms such as Bacillus licheniformis and Bacillus subtilis, but the gamma-polyglutamic acid according to the present disclosure is not limited to a synthesis or production method thereof or a type of microorganism. In the present disclosure, the gamma-polyglutamic acid may be comprised in the form of gamma-polyglutamic acid or a salt thereof.

In one embodiment, a weight average molecular weight of the gamma-polyglutamic acid or the salt thereof may be 10 to 10,000 kDa. Specifically, the weight average molecular weight of the gamma-polyglutamic acid or the salt thereof may be 10 to 100 kDa. When the weight average molecular weight of the gamma-polyglutamic acid or the salt thereof is less than 10 kDa, a moisturizing effect may be lowered, and when it exceeds 10,000 kDa, it may have an excessively high viscosity, or there is a limit to particulation, so that pilling due to a gel formulation not absorbed during skin application may occur.

In the present disclosure, “hyaluronic acid (HA)” is a linear high molecular polysaccharide in which β-D-N-acetylglucosamine and β-D-glucuronic acid are alternately bonded, and in the case of a human body, it is distributed in connective tissues such as subcutaneous tissue and cartilage tissue. Hyaluronic acid is excellent in biocompatibility, and although it is known to have useful effects on the skin such as improvement of moisturizing power of the skin, maintenance of skin elasticity, and improvement of skin barrier function, since structural stability is low and it is easily decomposed and removed in the body, research for stabilizing it has been conducted. Hyaluronic acid may be mainly biosynthesized by various types of microorganisms, but the hyaluronic acid according to the present disclosure is not limited to a synthesis or production method thereof or a type of microorganism. In the present disclosure, the hyaluronic acid may be comprised in the form of hyaluronic acid or a salt thereof.

In one embodiment, a weight average molecular weight of the hyaluronic acid or the salt thereof may be 10 to 10,000 kDa. Specifically, the weight average molecular weight of the high molecular hyaluronic acid or the salt thereof may be 500 to 5,000 kDa. When the weight average molecular weight of the high molecular hyaluronic acid or the salt thereof is less than 10 kDa, a moisturizing effect may be lowered, and when it exceeds 10,000 kDa, it may have an excessively high viscosity, or there is a limit to particulation, so that skin permeation may be difficult.

In one embodiment of the present disclosure, each salt of the gamma-polyglutamic acid and the hyaluronic acid is not particularly limited, but may be an inorganic acid salt, an organic acid salt, or an amino acid salt. For example, the salt may be inorganic acid salts such as hydrochloride, sulfate, hydroiodide, nitrate, and phosphate; organic acid salts such as citrate, oxalate, acetate, formate, propionate, sodium benzoate, maleate, tartrate, methanesulfonate, and benzenesulfonate; inorganic base salts such as sodium salt, potassium salt, calcium salt, magnesium salt, copper salt, zinc salt, aluminum salt, and ammonium salt; organic base salts such as triethanolammonium salt and pyridinium salt; and amino acid salts such as lysine salt, arginine salt, histidine salt, aspartate salt, and glutamate salt.

As one embodiment, the salt of the gamma-polyglutamic acid may be gamma-polyglutamic acid sodium salt or gamma-polyglutamic acid potassium salt.

As one embodiment, the salt of the hyaluronic acid may be hyaluronic acid sodium salt, hyaluronic acid potassium salt, hyaluronic acid acetate salt, hyaluronic acid ammonium salt, or C12-13 alkyl glyceryl hydrolyzed hyaluronate.

In one embodiment, the cross-linked gel may be, more specifically, a cross-linked gel in which a gel of gamma-polyglutamic acid or a salt thereof and a gel of hyaluronic acid or a salt thereof are cross-linked. That is, it may be a cross-linked gel in which after each of the two components is manufactured into a gel formulation, they are cross-linked.

In one embodiment, a weight ratio of the gamma-polyglutamic acid or the salt thereof, and the hyaluronic acid or the salt thereof comprised in the cross-linked gel may be more than 0.65 and less than 2:1. More specifically, the parts by weight of the gamma-polyglutamic acid or the salt thereof relative to 1 part by weight of the hyaluronic acid or the salt thereof may be more than 0.65 parts by weight, 0.7 parts by weight or more, 0.75 parts by weight or more, 0.8 parts by weight or more, 0.85 parts by weight or more, 0.9 parts by weight or more, 0.95 parts by weight or more, 1 part by weight or more, 1.05 parts by weight or more, 1.1 parts by weight or more, 1.15 parts by weight or more, 1.2 parts by weight or more, 1.25 parts by weight or more, 1.3 parts by weight or more, 1.35 parts by weight or more, 1.4 parts by weight or more, 1.45 parts by weight or more, 1.5 parts by weight or more, 1.55 parts by weight or more, 1.6 parts by weight or more, 1.65 parts by weight or more, 1.7 parts by weight or more, 1.75 parts by weight or more, 1.8 parts by weight or more, 1.85 parts by weight or more, 1.9 parts by weight or more, or 1.95 parts by weight or more, and may be less than 2.0 parts by weight, 1.95 parts by weight or less, 1.9 parts by weight or less, 1.85 parts by weight or less, 1.8 parts by weight or less, 1.75 parts by weight or less, 1.7 parts by weight or less, 1.65 parts by weight or less, 1.6 parts by weight or less, 1.55 parts by weight or less, 1.5 parts by weight or less, 1.45 parts by weight or less, 1.4 parts by weight or less, 1.35 parts by weight or less, 1.3 parts by weight or less, 1.25 parts by weight or less, 1.2 parts by weight or less, 1.15 parts by weight or less, 1.1 parts by weight or less, 1.05 parts by weight or less, 1 part by weight or less, 0.95 parts by weight or less, 0.9 parts by weight or less, 0.85 parts by weight or less, 0.8 parts by weight or less, 0.75 parts by weight or less, or 0.7 parts by weight or less. When the parts by weight of the gamma-polyglutamic acid or the salt thereof relative to 1 part by weight of the hyaluronic acid or the salt thereof is less than 0.65 parts by weight or 2 parts by weight or more, an effect of improvement in moisturizing for the skin and strengthening of skin barrier function may be lowered.

As one embodiment, a particle size of the cross-linked gel may be 20 to 300 μm. In the present specification, the particle size means the largest diameter of each particle, and means a particle size of at least 90% or more of particles distributed in the composition. Specifically, a particle size range of the particles may mean the largest intra-particle diameter range of at least 90% or more, 91% or more, 92% or more, 93% or more, 94% or more, 95% or more, 96% or more, 97% or more, 98% or more, or 99% or more of the particles distributed in the composition. More specifically, the particle size may be 20 μm or more, 50 μm or more, 100 μm or more, 150 μm or more, 200 μm or more, or 250 μm or more, and while being 300 μm or less, 250 μm or less, 200 μm or less, 150 μm or less, 100 μm or less, or 50 μm or less. A composition comprising the cross-linked gel particles according to one embodiment may provide an efficacy of increasing skin moisture content compared to a composition of a non-cross-linked formulation. When the particle size of the cross-linked gel according to one embodiment is less than 20 μm, skin moisturizing efficacy may be lowered, and when it exceeds 300 μm, a foreign body sensation and clumping phenomenon may appear during skin application.

Also, in one embodiment, an average particle size of the cross-linked gel may be 90 to 110 μm. The average particle size means an average value of the largest diameters of each particle, and means an average particle size of at least 90% or more of particles distributed in the composition. Specifically, the average particle size of the particles may mean an average particle size of at least 90% or more, 91% or more, 92% or more, 93% or more, 94% or more, 95% or more, 96% or more, 97% or more, 98% or more, or 99% or more of the particles distributed in the composition. More specifically, the average particle size may be 90 μm or more, 95 μm or more, 100 μm or more, or 105 μm or more, and while being 110 μm or less, 105 μm or less, 100 μm or less, or 95 μm or less.

In one embodiment, a content of the cross-linked gel may be 0.1 to 10 wt % based on a total weight of the composition. For example, the content of the cross-linked gel may be 0.1 wt % or more, 0.3 wt % or more, 0.5 wt % or more, 0.6 wt % or more, 0.7 wt % or more, 0.8 wt % or more, 0.9 wt % or more, 1.0 wt % or more, 1.1 wt % or more, 1.2 wt % or more, 1.3 wt % or more, 1.5 wt % or more, 1.7 wt % or more, 2 wt % or more, 3 wt % or more, 4 wt % or more, 5 wt % or more, 6 wt % or more, 7 wt % or more, 8 wt % or more, or 9 wt % or more based on the total weight of the composition, and may be 10 wt % or less, 9 wt % or less, 8 wt % or less, 7 wt % or less, 6 wt % or less, 5 wt % or less, 4 wt % or less, 3 wt % or less, 2.5 wt % or less, 2 wt % or less, 1.5 wt % or less, 1.3 wt % or less, 1.2 wt % or less, 1.1 wt % or less, 1 wt % or less, 0.9 wt % or less, or 0.8 wt % or less. When the content is less than 0.1 wt %, an effect on the skin may be insignificant, and when it exceeds 10 wt %, a viscosity is too high, so that a pilling phenomenon occurs during skin application and absorption, and thus a feeling of use may be lowered.

In one embodiment, the cross-linked gel may be a gel cross-linked by photo-cross-linking.

In one embodiment, the cross-linked gel may have a viscosity coefficient G″ of 200 Pa or more at a frequency of 10 Hz or more during a frequency sweep test using an Anton Paar MCR 302e rheometer. Specifically, the viscosity coefficient G″ may be 200 to 300 Pa at a frequency of 10 to 100 Hz during a frequency sweep test using an Anton Paar MCR 302e rheometer. The viscosity coefficient value is a value reduced by about 2 times or more than a hyaluronic acid or a salt thereof cross-linked gel not comprising gamma-polyglutamic acid or a salt thereof, and it means that a soft feeling of use may be provided when applying the composition to the skin since viscoelasticity and resistance are reduced through cross-linking of a gel of gamma-polyglutamic acid or a salt thereof and a gel of hyaluronic acid or a salt thereof.

The present disclosure is a manufacturing method of a composition according to the above-described embodiments, and may provide a manufacturing method comprising a cross-linked gel manufacturing step of manufacturing a cross-linked gel by photo-cross-linking a gel of gamma-polyglutamic acid or a salt thereof, and a gel of hyaluronic acid or a salt thereof at a weight ratio of gamma-polyglutamic acid or a salt thereof: hyaluronic acid or a salt thereof of more than 0.65 and less than 2:1.

In one embodiment, each of the gel of gamma-polyglutamic acid or a salt thereof and the gel of hyaluronic acid or a salt thereof may be a particulated gel. For example, an average particle size of each of the particulated gels may be 20 to 300 μm.

In one embodiment, the manufacturing method may further comprise, before the cross-linked gel manufacturing step, one or more of: a step of manufacturing a gel of gamma-polyglutamic acid or a salt thereof, and a step of manufacturing a gel of hyaluronic acid or a salt thereof.

As one embodiment, the step of manufacturing the gel of gamma-polyglutamic acid or the salt thereof is not limited as long as the gamma-polyglutamic acid or the salt thereof can be manufactured into a particulated gel formulation, but in one embodiment, the gel of gamma-polyglutamic acid or the salt thereof may be one manufactured without using a cross-linking agent.

As one embodiment, the step of manufacturing the gel of hyaluronic acid or the salt thereof is not limited as long as the hyaluronic acid or the salt thereof can be manufactured into a particulated gel formulation, but in one embodiment, the gel of hyaluronic acid or the salt thereof may be one manufactured without using a cross-linking agent.

As one embodiment, in the cross-linked gel manufacturing step, the photo-cross-linking may be photo-cross-linking by ultraviolet rays. More specifically, the cross-linked gel manufacturing step may comprise a step of putting the gel of gamma-polyglutamic acid or a salt thereof and the gel of hyaluronic acid or a salt thereof into a solvent and mixing and pulverizing the gels; and a step of cross-linking by irradiating the pulverized solution with ultraviolet rays.

In one embodiment, the photo-cross-linking may be cross-linking through 140 kGy electron beam irradiation. In one embodiment, an irradiation time of light during the photo-cross-linking may be 1 to 7 hours.

In one embodiment, the manufacturing method may further comprise, after manufacturing the cross-linked gel, a step of manufacturing a composition comprising the cross-linked gel as an active ingredient.

In one embodiment, the composition may be for skin moisturizing. In one embodiment, the composition may be for strengthening skin barrier function. In one embodiment, the composition may be for preventing skin elasticity reduction or for improving skin elasticity. In one embodiment, the composition may be for preventing skin wrinkle generation or for improving wrinkles. In one embodiment, the composition may be for improving or alleviating skin inflammation. Another embodiment of the present disclosure may provide a method for skin moisturizing, strengthening skin barrier function, preventing skin elasticity reduction or improving skin elasticity, preventing skin wrinkle generation or improving wrinkles, or improving or alleviating skin inflammation, comprising administering an effective amount of a cross-linked gel in which gamma-polyglutamic acid or a salt thereof, and hyaluronic acid or a salt thereof are cross-linked in a weight ratio of more than 0.65 and less than 2:1 to the skin of a subject in need thereof. Another embodiment may provide a use of a cross-linked gel in which gamma-polyglutamic acid or a salt thereof, and hyaluronic acid or a salt thereof are cross-linked in a weight ratio of more than 0.65 and less than 2:1, for manufacturing a composition for skin moisturizing, strengthening skin barrier function, preventing skin elasticity reduction or improving skin elasticity, preventing skin wrinkle generation or improving wrinkles, or improving or alleviating skin inflammation. Another embodiment may provide a cross-linked gel in which gamma-polyglutamic acid or a salt thereof, and hyaluronic acid or a salt thereof are cross-linked in a weight ratio of more than 0.65 and less than 2:1, for use in a composition for skin moisturizing, strengthening skin barrier function, preventing skin elasticity reduction or improving skin elasticity, preventing skin wrinkle generation or improving wrinkles, or improving or alleviating skin inflammation. Another embodiment may provide a non-therapeutic cosmetic use of a cross-linked gel in which gamma-polyglutamic acid or a salt thereof, and hyaluronic acid or a salt thereof are cross-linked in a weight ratio of more than 0.65 and less than 2:1 as an active ingredient for skin moisturizing, strengthening skin barrier function, preventing skin elasticity reduction or improving skin elasticity, or preventing skin wrinkle generation or improving wrinkles, or improving or alleviating skin inflammation.

In one embodiment, the composition may be a composition for external application to skin. In the present disclosure, the ‘composition for external application to skin’ means a composition of a formulation applied from the outside of the skin. As one embodiment, the composition for external application to skin may be applied by applying it directly by hand or by using various application means such as a spatula, and may comprise cosmetics, medicines, quasi-drugs, and the like of various formulations.

In one embodiment, the composition may be a cosmetic composition. In one embodiment, an appearance of the cosmetic composition may comprise a cosmetologically or dermatologically acceptable medium or base. For example, it may be provided in the form of a solution, a gel, a solid, a paste anhydrous product, an emulsion obtained by dispersing an oil phase in an aqueous phase, a suspension, a microemulsion, a microcapsule, a microgranule, or an ionic (liposome) and non-ionic vesicle dispersant, or in the form of a cream, a toner, a lotion, a powder, an ointment, a spray, or a conceal stick. These compositions may be manufactured according to conventional methods in the relevant field.

The cosmetic composition according to one embodiment of the present disclosure is not particularly limited in its formulation, and for example, it may be formulated into cosmetics such as an ampoule, a cream, a softening cosmetic water, an astringent cosmetic water, a nourishing cosmetic water, a nourishing cream, a gel cream, a massage cream, an essence, an eye cream, an eye essence, a cleansing cream, a cleansing foam, a cleansing water, a cleansing tissue comprising the cosmetic composition, a pack, a body lotion, a body cream, a body oil, and a body essence. When the formulation according to one embodiment of the present disclosure is a paste, a cream, or a gel, animal fibers, vegetable fibers, wax, paraffin, starch, tragacanth, cellulose derivatives, polyethylene glycol, silicone, bentonite, silica, talc, or zinc oxide and the like may be utilized as a carrier component. When the formulation according to one embodiment of the present disclosure is a spray, lactose, talc, silica, aluminum hydroxide, calcium silicate, or polyamide powder may be utilized as a carrier component, and additionally, a propellant such as chlorofluorohydrocarbon, propane/butane, or dimethyl ether may be comprised. In the case where the formulation according to one embodiment of the present disclosure is a solution or an emulsion, a solvent, a solvating agent, or an emulsifying agent is utilized as a carrier component, and for example, there are water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid esters of sorbitan. When the formulation according to one embodiment of the present disclosure is a suspension, a liquid diluent such as water, ethanol, or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tragacanth and the like may be utilized as a carrier component. When the formulation according to one embodiment of the present disclosure is a surfactant-containing cleansing, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamido betaine, aliphatic alcohol, fatty acid glyceride, fatty acid diethanolamide, vegetable oil, lanolin derivative, or ethoxylated glycerol fatty acid ester and the like may be utilized as a carrier component.

In the cosmetic composition according to one embodiment of the present disclosure, a functional additive and components comprised in a general cosmetic composition may be further comprised. As the functional additive, a component selected from the group consisting of water-soluble vitamins, oil-soluble vitamins, high molecular peptides, high molecular polysaccharides, sphingolipids, and seaweed extract may be comprised. As blending components comprised other than these, fat and oil components, a moisturizer, an emollient, a neutralizing agent, a surfactant, organic and inorganic pigments, organic powder, an ultraviolet absorber, an antiseptic, a bactericide, an antioxidant, a plant extract, a pH adjuster, alcohol, colorant, fragrance, a blood circulation promoter, a cooling agent, an antiperspirant, purified water, and the like may be mentioned.

In one embodiment, the composition may be a pharmaceutical composition. When the composition according to one embodiment of the present disclosure is applied to a medicine, it may be formulated into a parenteral administration agent in a semi-solid or liquid form by adding a carrier of an inorganic or organic type commonly used for the active ingredient used in one embodiment of the present disclosure, and the pharmaceutical composition according to the present disclosure may be administered parenterally, rectally, topically, transdermally, intravenously, intramuscularly, intraperitoneally, subcutaneously, and the like. As one embodiment, the pharmaceutical composition may be a parenteral administration agent, more specifically, an agent for external application to skin. For example, the agent for external application to skin may be a formulation such as an ointment, a skin, a lotion, a gel, a cream, a spray, a suspension, an emulsion, a patch, and the like, but is not limited thereto. The composition according to one embodiment of the present disclosure may easily formulate the active ingredient by being carried out according to a conventional method widely known in the art, and at this time, a surfactant, an excipient, a coloring agent, a spice, a preservative, a stabilizer, a buffer, a suspending agent, and other commonly used auxiliary agents may be appropriately used. An administration dose of the active ingredient of the pharmaceutical composition according to one embodiment of the present disclosure will vary according to the age, gender, weight, pathological state and severity thereof, administration route, or judgment of a prescriber of a subject to be administered. Determination of an appropriate dose based on these factors is within the level of those skilled in the art, and a daily administration dose thereof may be, for example, 0.1 mg/kg/day to 100 mg/kg/day, more specifically 5 mg/kg/day to 50 mg/kg/day, but is not limited thereto.

Hereinafter, the present disclosure will be described in more detail through the following examples. However, the following examples are provided only for the purpose of illustration to aid understanding of the present disclosure, and the scope and range of the present disclosure are not limited thereto.

### Manufacturing Example

As one embodiment according to the present disclosure, cross-linked gels of Example 1 and Comparative Examples 1 to 4 having the compositions of Table 1 below were manufactured by the following method.

Specifically, for polyglutamic acid gel (BP-Silky Gel), a polyglutamic acid raw material was quantified and dissolved in purified water. After dividing the solution and electron beam irradiation, water, butylene glycol, 1,2-hexanediol, and other additives were added. This was filtered through a 20 μm sieve to obtain a particulated gel having a uniform particle size.

For sodium hyaluronate gel (ECOBRIDGE HA), a sodium hyaluronate raw material was quantified and added to a mixture of a 0.24 N KOH solution and sodium trimetaphosphate (STMP) to be cross-linked and gelled. After neutralizing, washing, and pulverizing this, butylene glycol, 1,2-hexanediol, and other additives were added to obtain a particulated gel.

Each of the polyglutamic acid gel and the sodium hyaluronate gel is a raw material in which polyglutamic acid and sodium hyaluronate are water-dispersed in a particulated gel type, and each wt % in the composition of polyglutamic acid and sodium hyaluronate, which are simple linear polymers comprised in these gels, was converted and described together in the table below.

TABLE 1

| | Comparative | | Comparative | Comparative | Comparative |
|---|---|---|---|---|---|
| Component (%) | Example 1 | Example 1 | Example 2 | Example 3 | Example 4 |
| Purified water | To 100 | To 100 | To 100 | To 100 | To 100 |
| Polyglutamic acid | 0.5 | 0.67 | 1 | 1.35 | 1.52 |
| Sodium hyaluronate | 0.76 | 0.67 | 0.5 | 0.34 | 0.25 |
| 1,3-Butylene glycol | 4 | 4 | 4 | 4 | 4 |
| 1,2-Hexanediol | 2 | 2 | 2 | 2 | 2 |
| Chelating agent | Trace | Trace | Trace | Trace | Trace |
| Preservative | Trace | Trace | Trace | Trace | Trace |
| Total (wt %) | 100 | 100 | 100 | 100 | 100 |
| Weight ratio of | 0.65:1 | 1:1 | 2:1 | 4:1 | 6:1 |
| polyglutamic acid and | (1:1.5) | | | | |
| sodium hyaluronate | | | | | |

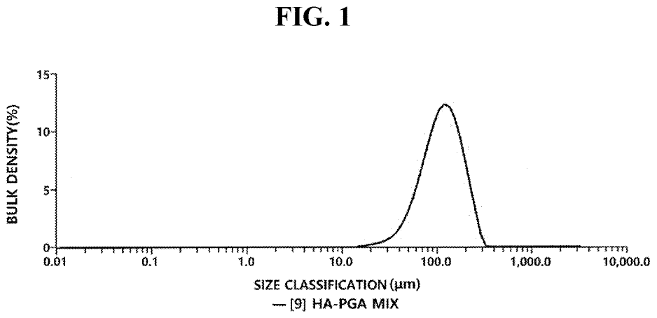

FIG. 1 is a view showing a result of analyzing a particle size distribution using a Mastersizer (Mastersizer 3000, Malvern Co.) in a water-dispersed state for the cross-linked gel of the polyglutamic acid gel and the sodium hyaluronate gel of Example 1. From FIG. 1, it can be confirmed that the composition according to one embodiment of the present disclosure comprises particles of a specific size of 2 to 300 μm.

### Test Example 1

Targeting Example 1 and Comparative Examples 1 to 4 manufactured in the Manufacturing Example, skin moisturizing effects were comparatively analyzed as follows.

First, a composition comprising 6.6 wt % was manufactured by diluting the cross-linked gel of each of Example 1 and Comparative Examples 1~4 in KGM® Gold Keratinocyte Growth Medium.

Normal human neonatal keratinocytes (HEKn, Lonza 192907) were cultured in a 6-well plate at 3×10⁵ cells/well. Specifically, KGM-Gold™ supplemented with a keratinocyte growth kit (Lonza, #192060) was cultured for 24 hours under incubator conditions of 37° C. and 5% CO₂. Then, the composition comprising the cross-linked gel of each of Example 1 and Comparative Examples 1~4 was treated on the cultured cells for 24 hours. Total RNA was isolated and analyzed with a BioAnalyzer 2100 (Agilent Technologies, Santa Clara, CA, USA). RNA was reverse-transcribed into cDNA through SuperScript® III reverse transcriptase (Invitrogen, Carlsbad, CA, USA). To determine an expression level of Caspase-14 as a target gene, a quantitative real-time TaqMan PCR technique was used (7500Fast, Applied Biosystems, Foster City, CA, USA).

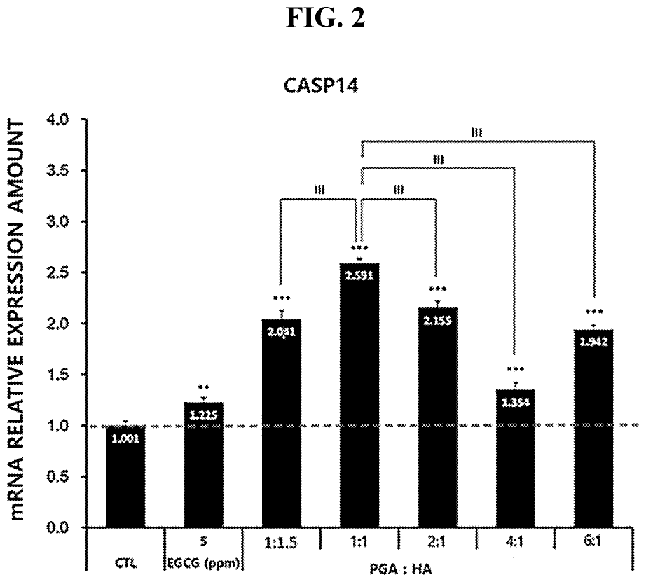

FIG. 2 is a view showing a result of analyzing a relative amount of CASP14 gene expression according to each composition of Example 1 and Comparative Examples 1-4, that is, a weight ratio of gamma-polyglutamic acid (PGA) and hyaluronic acid (HA). As a result of respectively treating cross-linked gels manufactured with PGA and HA at various concentration ratios, the CASP14 gene expression of Example 1, in which the weight ratio of polyglutamic acid (PGA) and hyaluronic acid (HA) is 1:1, was shown to be the highest, and based on this, the CASP14 gene expression was decreased in the case of 1:1.5 (0.65:1) and 2:1. Caspase-14 is one of enzymes that decompose filaggrin to generate a natural moisturizing factor NMF, and NMF has a property of absorbing moisture well, so it plays an important role in maintaining epidermal moisture and is decreased in dry skin. From the above results, as one embodiment of the present disclosure, it can be confirmed that a cross-linked gel in which a weight ratio of polyglutamic acid (PGA) and hyaluronic acid (HA) is cross-linked in a weight ratio of more than 0.65 and less than 2:1 contributes to maintenance of skin moisture and improvement of skin health by increasing expression of CASP14 protein, which plays a key role in strengthening skin barrier.

### Test Example 2

For rheological analysis of a cross-linked gel according to one embodiment of the present disclosure, the following experiment was performed.

First, each cross-linked gel of Example 1 and Comparative Example 5 according to the table below was manufactured according to the same method as the method described in the Manufacturing Example except for the weight ratio of gamma-polyglutamic acid and hyaluronic acid. After taking an amount suitable for a measurement geometry of a rheometer for each of the manufactured cross-linked gels, a frequency sweep range was set from 0.1 Hz to 100 Hz. Using an Anton Paar MCR 302e rheometer, a viscosity coefficient at room temperature or 35° C. was measured according to a cone-and-plate method.

TABLE 2

| | | Comparative |
|---|---|---|
| Component (%) | Example 1 | Example 5 |
| Purified water | To 100 | To 100 |
| Polyglutamic acid | 0.67 | 0 |
| Sodium hyaluronate | 0.67 | 1 |
| 1,3-Butylene glycol | 4 | 4 |
| 1,2-Hexanediol | 2 | 2 |
| Chelating agent | Trace | Trace |
| Preservative | Trace | Trace |
| Total (wt %) | 100 | 100 |
| Weight ratio of polyglutamic | 1:1 | 0:1 |
| acid and sodium hyaluronate | | |

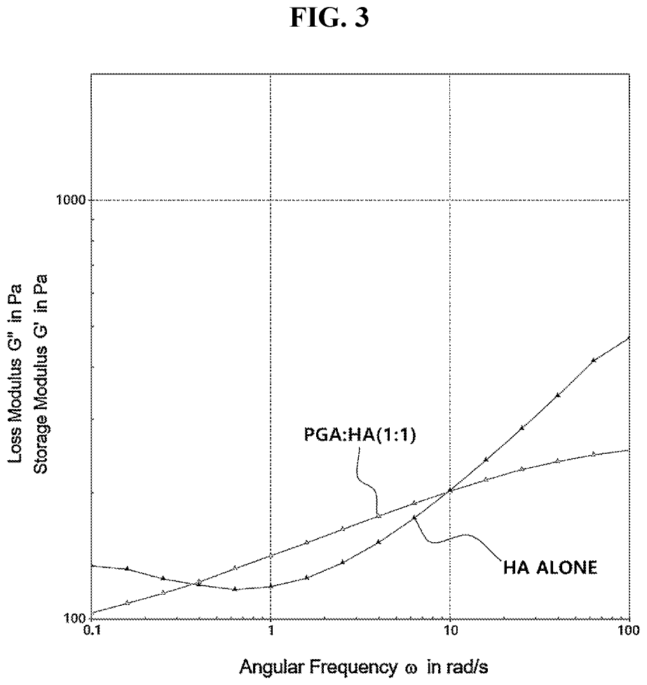

As a result, as shown in FIG. 3, Comparative Example 5 consisting of a hyaluronic acid (HA) cross-linked gel showed a tendency for a viscosity coefficient to significantly increase in a high frequency region during a frequency sweep test. This shows high viscoelasticity and resistance under high frequency, that is, fast deformation conditions, and suggests a potential problem that may lead to a pilling phenomenon in terms of an actual feeling of use. On the other hand, for a cross-linked gel comprising gamma-polyglutamic acid (PGA) and hyaluronic acid (HA) at 1:1 according to one embodiment of the present disclosure, the viscosity coefficient is 200 to 300 Pa, which is effectively decreased compared to Comparative Example 5, and thus it can be confirmed that a chronic feeling of use problem is improved and a unique soft finish is shown.

### Test Example 3

In order to comparatively analyze each feeling of use of Example 1 and Comparative Example 5 manufactured in Test Example 2, the following experiment was performed.

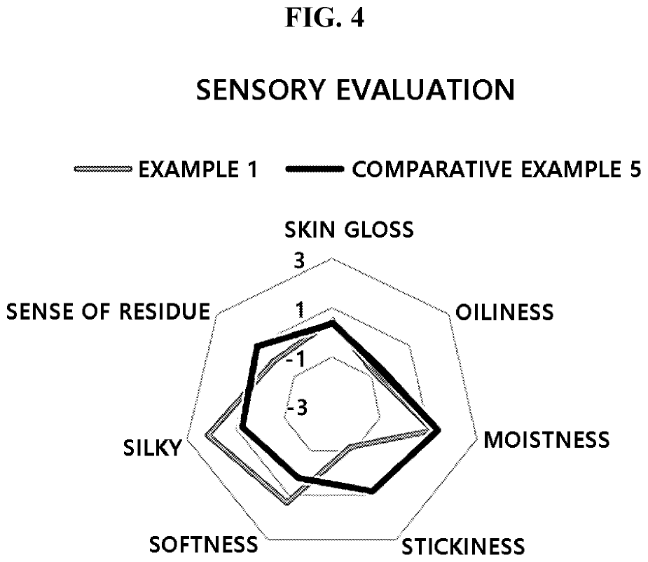

In this study, for 10 subjects consisting of 7 males and 3 females in their 30s-40s, compositions of Example 1 and Comparative Example 5 were quantified into sensory spider web data, and an average value was schematized and shown in FIG. 4. As a result of analysis, in the case of Example 1 in which a cross-linked gel comprising gamma-polyglutamic acid (PGA) and hyaluronic acid (HA) at 1:1 is comprised according to one embodiment of the present disclosure, decreased stickiness is shown compared to Comparative Example 5, while a sensory effect in which silky spreadability and softness are increased than Comparative Example 5 was confirmed. These results suggest that the composition (Example 1) according to one embodiment of the present disclosure can provide a more improved feeling of use.

### Test Example 4

In order to analyze skin moisturizing efficacy of a cross-linked gel according to one embodiment of the present disclosure, the following experiment was performed.

First, a composition comprising each cross-linked gel of Example 2 (HA:PGA=1:1 mixture), Comparative Example 5 (HA cross-linked gel alone), and Comparative Example 6 (PGA cross-linked gel alone) according to the table below was manufactured according to the same method as the method described in the Manufacturing Example except for the weight ratio of gamma-polyglutamic acid and hyaluronic acid.

TABLE 3

| | | Comparative | Comparative |
|---|---|---|---|
| Component (%) | Example 2 | Example 5 | Example 6 |
| Purified water | To 100 | To 100 | To 100 |
| Polyglutamic acid | 0.5 | 0 | 1 |
| Sodium hyaluronate | 0.5 | 1 | 0 |
| 1,3-Butylene glycol | 4 | 4 | 4 |
| 1,2-Hexanediol | 2 | 2 | 2 |
| Chelating agent | Trace | Trace | Trace |
| Preservative | Trace | Trace | Trace |
| Total (wt %) | 100 | 100 | 100 |
| Weight ratio of polyglutamic | 1:1 | 0:1 | 1:0 |
| acid and sodium hyaluronate | | | |

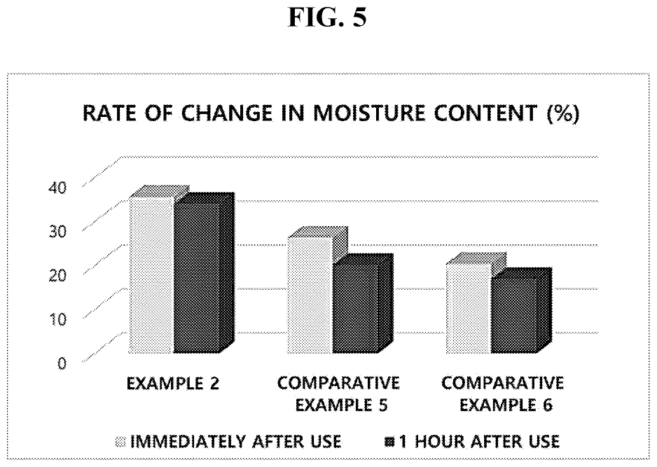

A composition corresponding to the Manufacturing Example was manufactured by diluting the cross-linked gel of each of Example 2, Comparative Examples 5 and 6 in distilled water, and for 5 adult males having normal skin, each of the manufactured compositions was applied to both forearm areas. Using a Cornemeter, an amount of change in skin moisture content at a point in time when 1 hour elapsed immediately after use was measured, and a rate of change (%) in skin moisture content is shown in FIG. 5.

As a result, Example 2 comprising a cross-linked gel comprising gamma-polyglutamic acid and hyaluronic acid in a weight ratio of 1:1 according to one embodiment of the present disclosure showed that skin moisture content was about 1.5 times to 2 times or more higher compared to Comparative Example 5 and Comparative Example 6, which are cross-linked gels comprising gamma-polyglutamic acid or hyaluronic acid alone, and it was confirmed that skin moisture loss after 1 hour of use was also significantly decreased, showing excellent skin moisturizing efficacy.

What is claimed is:

1. A composition comprising, as an active ingredient, a cross-linked gel in which gamma-polyglutamic acid or a salt thereof, and hyaluronic acid or a salt thereof are cross-linked in a weight ratio of more than 0.65 and less than 2:1.

2. The composition of claim 1, wherein a particle size of the cross-linked gel is 20 to 300 μm.

3. The composition of claim 1, wherein the cross-linked gel is comprised in an amount of 0.1 to 10 wt % based on a total weight of the composition.

4. The composition of claim 1, wherein the cross-linked gel has a viscosity coefficient G″ of 200 Pa or more at a frequency of 10 Hz or more during a frequency sweep test using an Anton Paar MCR 302e rheometer.

5. The composition of claim 1, wherein the cross-linked gel is a gel in which gamma-polyglutamic acid or a salt thereof, and hyaluronic acid or a salt thereof are cross-linked by photo-cross-linking.

6. A method for one or more of strengthening skin barrier function, skin moisturizing, preventing skin elasticity reduction or improving skin elasticity, and preventing or improving skin inflammation, comprising administering the composition of claim 1 in an effective amount to a subject in need thereof, or applying the same to skin of the subject.

7. The method of claim 6, wherein a particle size of the cross-linked gel is 20 to 300 μm.

8. The method of claim 6, wherein the cross-linked gel is comprised in an amount of 0.1 to 10 wt % based on a total weight of the composition.

9. The method of claim 6, wherein the cross-linked gel is a gel in which gamma-polyglutamic acid or a salt thereof, and hyaluronic acid or a salt thereof are cross-linked by photo-cross-linking.

10. The method of claim 6, wherein the cross-linked gel has a viscosity coefficient G″ of 200 Pa or more at a frequency of 10 Hz or more during a frequency sweep test using an Anton Paar MCR 302e rheometer.

11. A manufacturing method of the composition of claim 1, the manufacturing method comprising a cross-linked gel manufacturing step comprising photo-cross-linking a gel of gamma-polyglutamic acid or a salt thereof, and a gel of hyaluronic acid or a salt thereof at a weight ratio of gamma-polyglutamic acid or a salt thereof: hyaluronic acid or a salt thereof of more than 0.65 and less than 2:1.

12. The manufacturing method of claim 11, wherein the cross-linked gel manufacturing step is to manufacture the cross-linked gel to have a particle size of 20 to 300 μm.

13. The manufacturing method of claim 11, wherein the cross-linked gel manufacturing step is to manufacture a cross-linked gel having a viscosity coefficient G″ of 200 Pa or more at a frequency of 10 Hz or more during a frequency sweep test using an Anton Paar MCR 302e rheometer.

14. The manufacturing method of claim 11, further comprising:
before the cross-linked gel manufacturing step,
one or more steps of:
a step of manufacturing a gel of gamma-polyglutamic acid or a salt thereof, and
a step of manufacturing a gel of hyaluronic acid or a salt thereof.

15. The manufacturing method of claim 11, wherein the cross-linked gel manufacturing step comprises a step of putting the gel of gamma-polyglutamic acid or a salt thereof and the gel of hyaluronic acid or a salt thereof into a solvent and mixing and pulverizing the gels; and a step of cross-linking by irradiating the pulverized solution with ultraviolet rays.
